(19) United States
(12) Patent Application Publication
(10) Pub. No.: US 2018/0326144 A1
(43) Pub. Date: Nov. 15, 2018

(54) ENDOSCOPE SYSTEM AND METHOD OF USE

(71) Applicant: Meditrina, Inc., Cupertino, CA (US)

(72) Inventor: Csaba Truckai, Saratoga, CA (US)

(73) Assignee: Meditrina, Inc., Cupertino, CA (US)

(21) Appl. No.: 15/975,626

(22) Filed: May 9, 2018

Related U.S. Application Data
(60) Provisional application No. 62/506,504, filed on May 15, 2017.
(60) Provisional application No. 62/505,763, filed on May 12, 2017.

(51) Int. Cl. A61M 3/02; A61B 1/015; A61B 1/303; A61B 34/20; A61M 31/00; A61B 1/018; A61B 18/14

(52) U.S. Cl. CPC A61M 3/0254; A61B 1/015; A61B 1/303; A61B 34/20; A61B 2017/4216; A61M 31/00; A61B 1/018; A61B 18/1485; A61M 3/0279

(57) ABSTRACT

A fluid management system is with a fluid source, a fluid receptacle, and a surgical tool having an inlet port for receiving fluid from the fluid source and an outlet port for discharging fluid to the fluid receptacle. The fluid management system includes a pump head assembly and a tubing cassette removably insertable over the pump head assembly. The pump head assembly includes an inlet pump and an outlet pump, where each pump has a rotor, a plurality of rollers, an arcuate roller backing adjacent the rotor to define a tube receiving channel therebetween, and a rotor position sensor. An inlet motor configured to rotate the inlet pump rotor, and an outlet motor configured to rotate the outlet pump rotor. The tubing cassette includes an inlet tubing loop and an outlet tubing loop, where each tubing loop is received about the rollers in the tube receiving channel of the inlet pump when the cassette is placed over the pump head assembly. A controller receives rotational position information from the rotor position sensor of each pump and instructs the motor of each pump to drive said rotor and selectively stop each rotor at a preselected rotational position.

### CROSS-REFERENCE TO RELATED APPLICATION

This application claims the benefit of provisional application no. 62/506,504 (Attorney Docket No. 50553-710.101), filed on May 15, 2017, and of provisional application no. 62/505,763 (Attorney Docket No. 50553-709.101), and filed on May 12, 2017, the full disclosures of which are incorporated herein by reference.

### BACKGROUND OF THE INVENTION

1. Field of the Invention. The present invention relates generally to medical devices and methods, and more particularly to an endoscopic viewing system configured for hysteroscopy.

Endoscopic systems intended for hysterectomy typically comprise a base station having an image display, a disposable endoscope component with an image sensor, a re-usable handle component that includes an image processor and transceiver configures for wireless communication and image transmission with the base station, and a fluid management system integrated with or in wireless communication with the base station and handle component.

Of particular interest to the present invention, a fluid management system intended for use with a hysteroscopic endoscope may comprise a pump head assembly having a pair of pumps, one for delivering fluid from a fluid source to the endoscope and the other for receiving spent fluid from the endoscope and transferring the fluid to a collection vessel. The pumps will often comprise rotors having rollers which are used together with feed tubes to form peristaltic or “tube” pumps. The tubes are often combined in an enclosure as a “tubing cassette” where the tubing cassette is disposable and isolates the pump head assembly from exposure to the fluids.

While the use of disposable tubing cassettes and reusable pump head assemblies has been very effective, the nature of the pump rotors can make insertion and removal of the tubing cassette difficult. For example, a random orientation of the rollers on the pump rotors can make mounting and removal of the pump cassette very unpredictable and, in certain circumstances, difficult to perform. In some cases, the user has had to reposition the pump rotors more than once before being able to conveniently insert or retrieve the tubing cassette on the pump head assembly.

For these reasons, it would be desirable to provide improved systems and methods for inserting and removing tubing cassettes onto the pump head assemblies of fluid management systems of the types used in hysteroscopic and other medical procedures. At least some of these objectives will be met by the inventions described hereinbelow.

### SUMMARY OF THE INVENTION

In a first aspect, the present invention provides a fluid management system for use with a hysteroscopic or other medical endoscopic system. Typically, the hysteroscopic or other endoscopic system will include a fluid source, a reservoir or receptacle for collecting returned fluid, and a surgical tool having an inlet port for receiving fluid from the fluid source and an outlet port for discharging fluid to the fluid reservoir. The fluid management system of the present invention typically comprises a pump head assembly and a tubing cassette. The pump head assembly comprises an inlet or inflow pump having a rotor with a plurality of rollers, an arcuate roller backing adjacent the rotor to define a tube-receiving channel therebetween, and a position sensor. The pump head assembly also comprises an outlet or outflow pump having a rotor with a plurality of rollers, an arcuate roller backing adjacent the rotor to define a tube-receiving channel therebetween, and a position sensor. An inlet motor is configured to rotate the inlet pump rotor and an outlet motor is configured to rotate the outlet pump rotor.

The tubing cassette, in turn, comprises an inlet or inflow tubing loop configured to be received about the rollers in the tube-receiving channel of the inlet pump when the cassette is placed over the pump head assembly. Similarly, an outlet or outflow tubing loop is configured to be received about the rollers in the tube-receiving channel of the outlet pump when the cassette is placed over the pump head assembly. Additionally, a controller is configured to receive rotational position information from the rotor position sensor of each pump and to instruct the motor of each pump to drive said rotor and selectively stop said rotor at a pre-selected rotational position, in one case where the rollers on the rotors are positioned in a manner that facilitates insertion and removal of the tubing cassette on and off from the pump head assembly.

In specific aspects of the fluid management system, the sensor may comprise an encoder, typically an encoder which is formed integrally in each motor of the pumps. In other instances, the encoder could be mounted externally on the motor or rotor. The controller is usually configured to selectively stop at least one rotor so that one roller on that rotor is aligned with a center line of the arcuate roller backing of that pump. Usually, the controller is further configured to selectively stop both rotors so that each rotor has a roller aligned with the center line of the adjacent roller backing. Typically, the pumps and the controller will be housed together in a single enclosure, such as a base unit or a station having other components of the endoscopic system therein.

In a second aspect, the present invention provides a method for loading a tubing cassette onto a pump head assembly. The method comprises providing a pump head assembly and a tubing cassette, each typically having at least certain of the features described hereinabove. The rotors on the pump head assembly are caused to each align one roller with a center line of the adjacent arcuate backing and typically further to assure that no other rollers are in the tube-receiving channel. In this way, the tubing cassette may be placed over the pump head assembly so that both the inlet tubing and the outlet tubing are received in the tube-receiving channels of the inlet and outlet pumps, respectively, with minimum difficulty. This particular positioning of the rollers minimizes interference from the individual rollers and, most advantageously, will often be made to occur automatically so that no intervention by the user is necessary to facilitate insertion or removal of the tubing cassette. In other instances, however, the user may be able to press a button or switch, or use voice activation, in order to initiate an otherwise automatic alignment of the pump rotors prior to insertion and/or removal of the tubing cassettes.

In specific aspects of the methods herein, causing the rotors on the pump head to each align one roller with the center line of the adjacent arcuate backing usually comprises sensing a position of the rotors and automatically aligning the rotors as noted. For example, the rotors may be automatically aligned each time the cassette is removed from the pump head so that they will be properly aligned next time the cassette is to be introduced. Usually, such automatic alignment is performed by a controller which is programmed to perform these method steps. Alternatively, in some instances the controller can be manually instructed or triggered to automatically align the rotors. For example, a user could press a button on a control panel near the pump head assembly to assure that proper alignment is achieved immediately before placement of the cassettes on the pump head assembly. Still further alternatively, the rotors may be automatically aligned each time the pump head assembly is powered, i.e. turned on for use. Typically, sensing the position of the rotors comprises reading an encoder coupled to the motor or to the rotor.

### BRIEF DESCRIPTION OF THE DRAWINGS

Various aspects of the invention will become clear from the following description of an illustrative embodiment and from the attached drawings, in which:

FIG. 1 is a schematic view of an endoscopic viewing system of the invention including a base station and image display, a single use endoscope component coupled to re-usable handle component, a fluid management system and a treatment tool introduced through a working channel in the endoscope component.

FIG. 2 is a perspective exploded view of the handle and endoscope components of the system of FIG. 1.

FIG. 3 is another perspective view of handle and endoscope components of FIG. 2.

FIG. 4 is a perspective view of a distal portion of the endoscope shaft of FIGS. 1 and 2 including a resilient, deformable distal portion that carries an image sensor, LEDs and collapsible working channel showing the distal portion in a straight insertion configuration.

FIG. 5A is a longitudinal sectional view through a portion of the shaft and the distal deformable portion of the endoscope in an insertion configuration with a collapsed working channel.

FIG. 5B is another longitudinal sectional view similar to that of FIG. 5A with the distal portion in a deformed or displaced configuration after being deflected by a rigid tool shaft inserted through the non-collapsed working channel.

FIG. 6 is a sectional view of an extrusion of a rigid portion of the endoscope shaft taken along line 6-6 of FIG. 5A.

FIG. 7A is a view of the proximal hub of the single-use endoscope component showing a pressure transducer component.

FIG. 7B is a view of the proximal hub of FIG. 7A coupled to the handpiece component showing a redundant pressure sensors therein.

FIG. 8 is a perspective view of the distal portion of the single-use endoscope component showing a pressure sensing channel.

FIG. 9 is a schematic view of a fluid management cassette and dual peristaltic pumps of the fluid management system of FIG. 1.

FIG. 10A is a schematic view of the deformable working channel component in a repose, non-tensioned position.

FIG. 10B is a view of the deformable working channel of FIG. 10A in a tensioned position as if an articulating shaft tool was introduced through the channel.

### DETAILED DESCRIPTION OF THE INVENTION

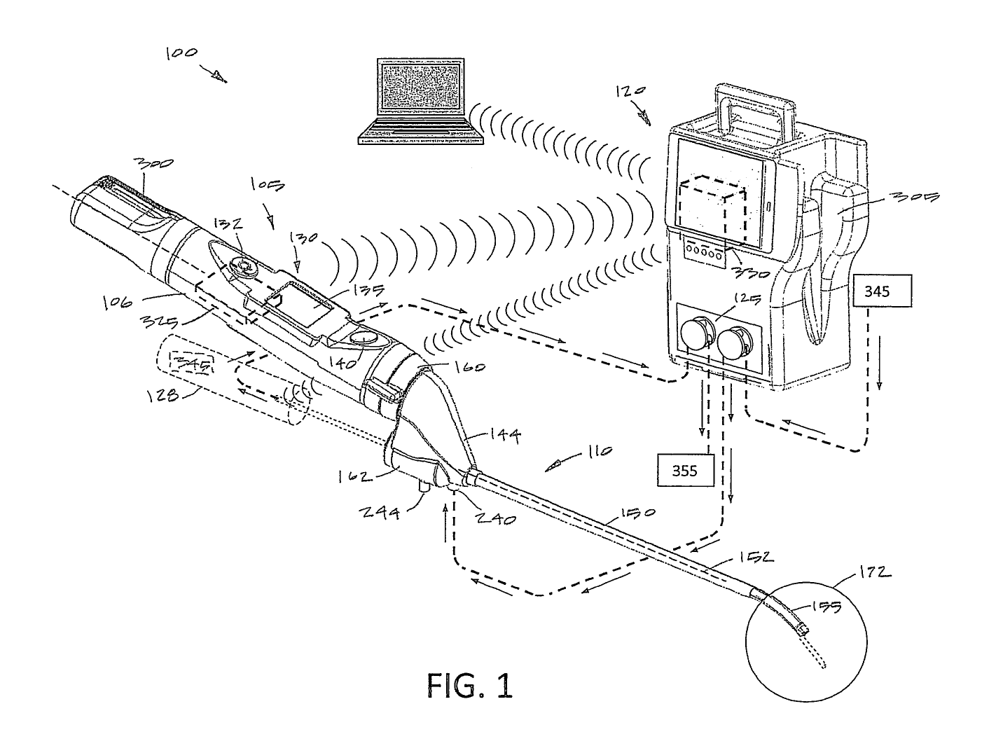

FIGS. 1 illustrates an endoscopic viewing system 100 corresponding to the invention which comprises several devices, components and communication links, typically including (i) a endoscope 105 consisting of a reusable handle component 106 that is detachably coupled to disposable endoscope shaft component 110; (ii) a base unit or station 120 that may include a controller and image processor together with a video/touch screen; (iii) a fluid management system 125 which is adapted for circulation of a distention fluid through a body cavity; (iv) a surgical tool 128 (phantom view) that may be introduced through the endoscope shaft component 110 for performing a therapeutic procedure in the interior of a patient's body; and (v) wireless communication links between the handle component 106 and the base station 120 that are capable of transmitting images and also capable of transmitting and receiving operating signals for controlling all operating parameters of the fluid management system 125, an electrosurgical tool, a motor driven tool, or other energy delivery features of any tool as will be described further below. While the system 100 is described herein for use in hysteroscopy, the system or a variation thereof can be used for various endoscopic procedures.

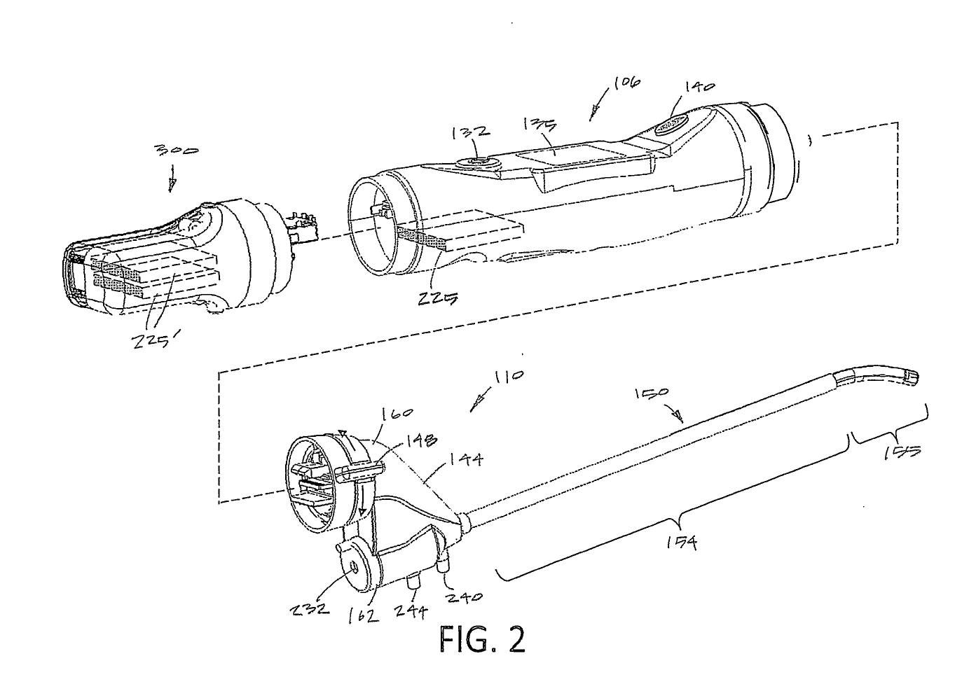

In FIGS.1-2, it can be seen that the endoscope 105 has a handle component 106 that is adapted for gripping with a human hand a control panel 130 that can be finger operated by the physician. An ON/OFF button 132 and a touch screen display 135 are provided in an upper surface of the handle 106. The touchscreen 135 can be used for adjusting operating parameters of endoscope 105 and the fluid management system 125 as well as for other operations. Various forms of touchscreens can be used with variations described further below. A lock/unlock button 140 is shown in the upper surface of the handle 106 in FIG. 1. The lock/unlock button 140 serves the function of preventing in the inadvertent adjustment of an operating parameter during use of the device by touching the touchscreen 135 by accident. The lock/unlock button 140 can take various forms, and in one variation can be pushed by the physician to lock in selected operating parameters after such parameters were selected on the touchscreen. In another variation, the selected operating parameters can be automatically locked after a time delay from 1 seconds to 5 seconds or more after such parameters are selected. In this variation, the lock/unlock is depressed to allow the physician to adjust operating parameters on the touchscreen.

Referring to FIGS. 1, 2 and 3, it can be seen that the handle component 106 is detachably coupled to the disposable endoscope shaft component 110. The endoscope shaft component 110 includes a proximal hub 144 that is adapted for coupling to the distal end 146 of the handle component 106 with a latch mechanism 148 carried by the hub 144 for locking the hub to the handle. The endoscope component 110 has an elongate shaft 150 extending along longitudinal axis 152. The shaft 150 includes a substantially rigid proximal portion or sleeve 154 that extends to a flexible, resilient distal shaft portion 155 described further below. It can be seen in FIGS. 1-2 that the proximal hub 144 has an offset connector portion 160 that is angled relative to the axis 152 for connecting to handle component 106. The proximal hub 144 of the endoscope component 110 as a tool-receiving portion 162 that is aligned with a working channel 165 extending through the elongate shaft 150. Thus, it can be understood how the physician may grip the handle component 106 with one hand and introduce a tool 128 through the working channel 165 and into a targeted working space 172 in the interior of a patient's body (FIG. 1).

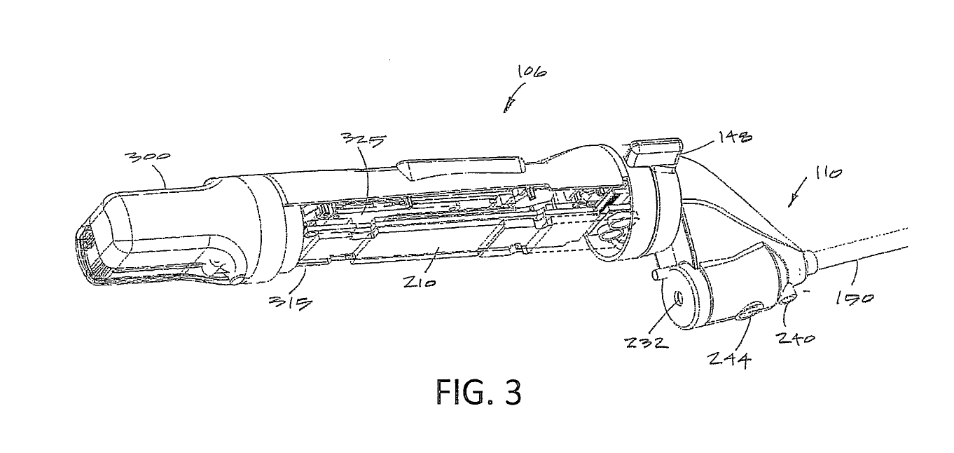

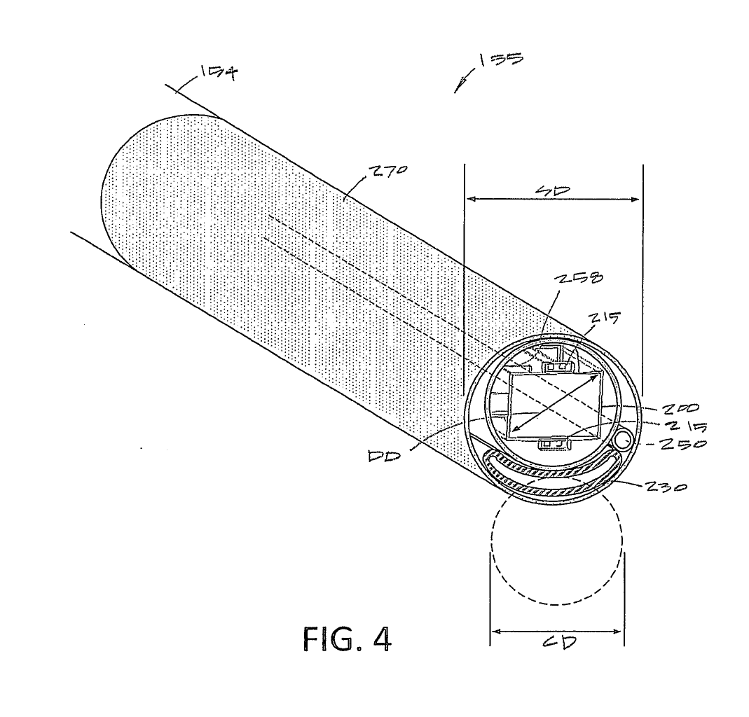

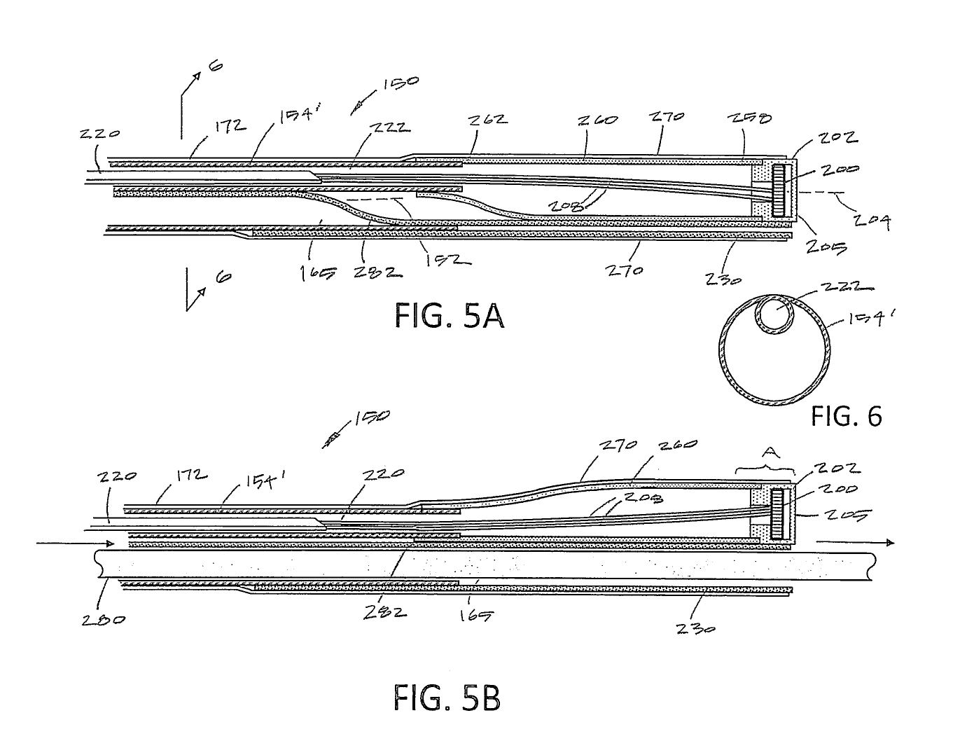

Now referring to the FIGS. 2, 4 and 5A, the elongate shaft 150 extends to a distal flexing or deformable portion 155 that carries an electronic image sensor 200. In one variation, the image sensor 200 is carried in a support member 202 which supports the sensor's viewing in axis 204. The support member 202 is covered by a transparent tip member 205 (not shown in FIG. 4) that can be seen in FIG. 5A. The transparent tip 205 can further comprise a focusing lens and/or a prism for modifying the sensor's field of view. The image sensor 200 is further operatively connected by leads 208 in the shaft 150 to an image processor 210 carried in a handle component 106 (FIG. 3). Alternatively, the image processor 210 or components thereof can be carried in the base unit 120 (FIG. 1). In FIG. 3, at least two LEDs 215 are provided for illuminating a working space in the patient's body. Alternatively, the electrical leads for the image sensor 200 can be coupled to the image processor 210 by conductors in an elongated flex circuit 220 extending through passageway 222 in the shaft 150. Similarly, wire leads (not shown) in such a flex circuit can connect the LEDs to an electrical source or battery 225 in the handle component 106 (FIG. 2).

Referring to FIGS. 2, 4 and 5A, in one variation, shaft 150 has a diameter ranging between 2.5 mm and 6 mm with a length configured for use in hysteroscopy. More commonly, the shaft diameter is from 4 mm to 6 mm in diameter. As will be described below, the shaft 150 is configured with a working channel 165 that may have a diameter ranging between 1 mm and 6 mm in the proximal shaft portion 154 that transitions to a collapsible portion 230 of working channel 165 (FIGS. 4, 5A) in the distal shaft portion 155. The working channel or tool-receiving channel 165 is adapted for receiving various types of tools. For example, a biopsy device may have a flexible shaft (not shown) with a diameter ranging from 1 mm to 3 mm and can be introduced through port 232 (FIGS. 1-2) in the proximal hub 144 which extends through channel 165 in the shaft 150 to the working end. Alternatively, a tissue resecting device (see FIG. 5B) can be used which may have a larger rigid shaft with a diameter, for example, from 2.5 mm to 5 mm. Such a rigid shaft tool similarly may be introduced through port 232 as shown in FIG. 5B.

Still referring to FIGS. 1-3, in one variation, the proximal hub 144 of the endoscope component 110 has first port 240 that communicates with a fluid flow channel 242 that extends through the elongate shaft 150 to an open termination in a distal region of the shaft 150. The proximal hub carries a second port 244 that communicates with the working channel 165 for providing a second flow passageway through the shaft portion 150. As will be described below, the first and second flow pathways can be used either for inflows for outflows. Referring to FIG. 3, the third flow passageway 250 extends to the shaft 150 to the proximal hub 144 and the pressure sensing mechanism as will be described in more detail below. In one variation, the third flow passageway 250 (FIG. 4) comprises a static fluid channel when filled with saline or distention fluid and thus can accurately measure actual pressure in a working space. The ports 240 and 244 coupled to the flow channels can be configured with Luer fittings or other types of connectors connector in the hub 144. In one variation, the fluid management system 125 can be coupled to ports 240 and 244 to provide fluid inflow and outflow channels in the endoscope to provide a circulating flow through a patient's uterine cavity and then can maintain a set intra-cavity pressure as is known in the art.

Now referring to FIGS. 2, 4 5A and 6, the perspective and cut-away views show the proximal shaft portion 152, the distal shaft portion 155 and the assembly of components that carry the image sensor 200 and the collapsible portion 230 of the working channel 165. In FIG. 2, the substantially rigid proximal shaft portion 154 can comprise a metal or plastic and in one variation is an extruded polymer as shown in FIG. 6 that has an interior wall 254 that provides an interior passageway 222 that carries the electrical leads or flex circuit 220 to the image sensor 200 and LEDs 215. In a typical embodiment, the flexible distal shaft portion 155 is curved for hysteroscopy as shown in FIGS. 1-2, but it should be appreciated that the distal shaft can be straight as shown for convenience in FIGS. 4 and 5A. The image sensor 200 is carried at a distal end of distal shaft portion 155 with a transparent distal tip 205 shown in FIG. 5A that can comprise a clear material such as a plastic lens material which is sealed and coupled to the support member 202. The support member 202 is coupled to the distal end 258 of a flexible polymer sleeve 260 which can have a repose curved shape as shown in FIGS. 1-2. The polymer sleeve 260 typically is not elastomeric but flexible and has a proximal end 262 that is necked down for a fixed coupling to the rigid shaft portion or sleeve 154 as shown in FIG. 5A. The distal shaft portion 155 includes a collapsible polymer sleeve 230 through which the working channel 165 extends. In FIG. 4, it can be seen of the polymer sleeve 230 is in a collapsed position.

FIGS. 4 and 5A further show that the entire distal shaft portion 155, including polymer sleeve 260 and polymer working channel sleeve 230 are constrained within an outer elastomeric sleeve 270. Thus, the outer elastomeric sleeve 270 is in a non-stretched or non-tensioned position which will collapse the working channel sleeve 230 as shown in FIG. 4. As can be seen in FIGS. 4 and 5A, the proximal end 172 elastomeric sleeve 170 is fixed with adhesives or by other means to the rigid proximal sleeve member 154′.

In FIG. 5A, it can be seen that the endoscope shaft 150 and elastomeric sleeve 170 are in an insertion profile or configuration wherein the elastomeric sleeve 170 is in a repose, non-tensioned position and working channel 165 is effectively closed in the distal end of the shaft as the elastomeric sleeve 170 is collapsed working channel sleeve 230.

Now turning to FIG. 5B, it can be seen that when the physician inserts an elongate rigid shaft 280 of tool 128 (FIG. 1) through the working channel 165, it will interface with the wall 282 of the repose, collapsed sleeve 230 in a distal portion of the device (see FIG. 5A) and then open and straighten the working channel sleeve 230 which thereby stretches the outer constraining elastomeric sleeve 270. In other words, advancement of the tool shaft 282 through the working channel 165 and straighten the working end of the tool exits the open termination 284 of working channel 165. As also can be seen in FIG. 5B, the image sensor 200 is moved in displaced radially outward from the central axis 152 of shaft 150, however the length A of the sensor support member 202 is configured to insure that the viewing axis 204 of the image sensor 200 is still at a selected angle, for example perpendicular to the axis 152 of shaft 150. The length A of the sensor support member 202 can range from 1 mm to 10 mm. When the shaft 280 of the tool 128 is withdrawn from the working channel 165, the elastomeric sleeve 170 will return from the tensioned position of FIG. 5A to the repose or non-tensioned position of FIG. 5A to provide the insertion/withdrawal profile of the shaft 150.

In general, referring to FIG. 4, the endoscope corresponding to the invention allows for the use of an image sensor 200 having a large diagonal dimension DD relative to the insertion profile or shaft diameter SD of the endoscope shaft 150 while at the same time providing a working channel 165 that has a large channel diameter CD relative to the insertion profile or diameter SD of the endoscope shaft 110. More in particular, the endoscope comprises a shaft 150 having a shaft diameter SD extending about a longitudinal axis 152 to a distal portion 155, an image sensor 200 with a diagonal dimension DD carried by the distal portion 155, and a working channel having a diameter CD extending through the shaft and distal portion, wherein the working channel portion in the distal region of the shaft is adjustable in shape to accommodate a tool introduced therethrough and wherein the combined sensor's diagonal dimension DD and the channel diameter CD is greater than the shaft diameter SD (see FIG. 4). In a variation, the sensor diagonal dimension DD is greater than 50% of the shaft diameter SD, greater than 60% of the shaft diameter or greater than 70% of the shaft diameter. In a variation, the working channel diameter CD is greater than 30% of the shaft diameter, greater than 40% of the shaft diameter or greater than 50% of the shaft diameter. In addition, the working channel portion in the distal region of the endoscope is adjustable between a curved shape and a straight shape. In a variation, the working channel portion in the distal region of the endoscope is adjustable between an at least partially collapsed shape and a non-collapsed shape.

In another aspect of the invention, the image sensor 200 can be carried in a non-orthogonal position relative to the longitudinal axis of the shaft 150 to orient the sensor's field of view to be aligned with a working space 172 distal from the end of the endoscope after a tool is inserted through the working channel 165. In a variation, the image sensor 200 is a support member at an angle ranging between 45° to 90° relative to the longitudinal axis 152 of the proximal shaft portion 154 to provide a selected field of view.

While FIGS. 4 and 5A illustrated a single collapsible sleeve 230, it should be appreciated that more than one collapsible sleeve in the interior channel can be provided in the endoscope shaft and constraining by an outer elastomeric sleeve 270.

Now turning to FIGS. 1, 2 and 3, it can be seen that the handle portion 106 includes a detachable proximal component 300 that carries a battery life extension pack. In FIG. 2, the battery 225 is provided for operating image processor and the wireless transmitting and receiving components that are further described below. The battery can be a lithium-ion battery or any other suitable form a battery. In the form factor the handle 106, such a battery 225 can provide from 10 minutes to 60 minutes of operating time. In one variation, the detachable proximal component 300 carries from 1 to 4 or more additional batteries 225′ which can provide additional operating time again ranging from 10 to 60 minutes per battery 225′. In one aspect of the invention, by providing at least one battery 225 in the handle portion 106, with additional batteries in a detachable component 300, the system allows for hot-swapping the batteries, that is, the first battery 225 in the handle portion 106 can maintain operating function while the detachable component 300 swapped out.

Following a procedure, the base station 120 includes a docking station 305 in which the handle 106 can be cradled which further is wirelessly coupled to a charger for charging the batteries 225 and 225′ as is known in the art.

Now turning to FIG. 3, it can be seen that interior space 315 of the handle portion 106 carries an image processor indicated at 210. That interior space 315 further carries a wireless transceiver 325 that is adapted to wirelessly communicate with the transceiver 330 in the base station 120. Thus, the control panel 130 in the handle 106 can be used to adjust operating parameters of the system and the transceiver 325 can wirelessly communicate with the base station, for example, to operate the fluid management system 125. In one variation, the control panel 130 and touch screen 135 can be used to set pressure in the working space 172, such as a patient's uterine cavity. Further, the touchscreen 135 can be used to wirelessly communicate with the base station 120 and fluid management system 125 to operate the inflow and outflow peristaltic pumps 340A and 340B. The inflow pump 340A provides fluid inflows from fluid source 345 which can be a saline bag as is known in the art. The outflow pump 340B provides fluid outflows from the working space wherein the fluid is collected in a collection reservoir 355.

In another variation, referring to FIG. 1, the control panel 130 and touchscreen 135 on the handle portion 106 can wirelessly communicate with the base station transceiver 330 which in turn can wirelessly communicate with a cooperating transceiver 345 in the tool 128 to adjust operating parameters of the tool. In one example, the tool 128 may have a motor drive which is operated to actuate a cutting device or other treatment device at the distal end of the tool shaft. In another example, the wireless control system may be used to adjust the electrosurgical or other energy delivery operating parameter of the tool 128. For example, an RF tool may be turned on and off, switch between an ablation and coagulation waveform, pulsed or the like.

In another aspect of the invention, referring to FIG. 3, the image processor 210 and associated microprocessors are configured for wirelessly transmitting data that supports various imaging sensors 200 that may be used in different disposable components 110. For example, some disposable endoscopic components 110 may carry a CMOS or image sensor 200 with a 1080p resolution, while other disposable endoscopic components may use a 720p sensor, a 200×200 sensor or a 400×400 senor, etc. Thus, the disposable component 110 will have a device identifier element that can be read by the handle component 106 when both components are assembled wherein the processing capabilities in the handle 106 will then identify and select the appropriate processor algorithms for the identified image sensor and for wirelessly transmitting the data.

In another aspect of the invention, referring to FIGS. 1-3, the system 100 will further include a device pairing mechanism that will definitively pair the handle portion 106 with the base station 120 to ensure that the handle transceiver 325 only wirelessly communicates with the associated base station 120, and not another base station that may be located within close proximity to the particular handle portion 106 being used. In one variation, the system could include a lockout mechanism to prevent operation of the system until the physician move the handle 106 into a particular docking portion of the base station to provide a so-called handshake between the two components. In another variation, each handle portion 106 can be adapted to send data packets with device identification to the base station which would be preprogrammed to only read data packets from the particular handle portion in use. This aspect of the invention is important for future use in operating environments where multiple handle portions 106 and base stations may be used in adjacent operating suites wherein secure point-to-point was communication links are required.

In another aspect of the invention the system includes upgradable firmware such that a peripheral device can “push” software updates to all the paired devices, for example, with updates directed to a base station 120, and then from the base station to a handle portion 106, or directly to the handle portion.

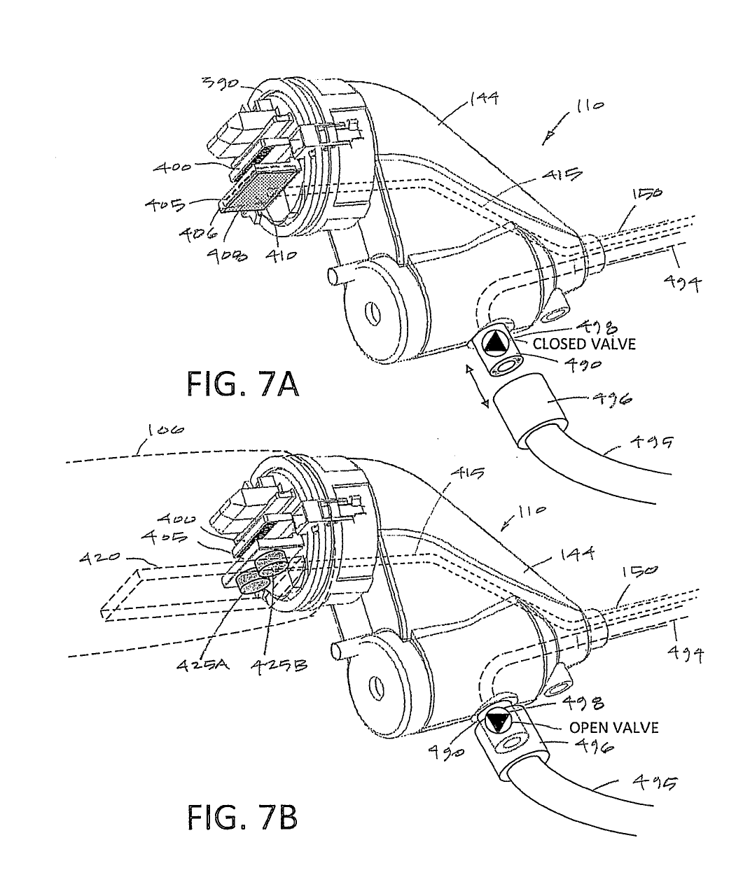

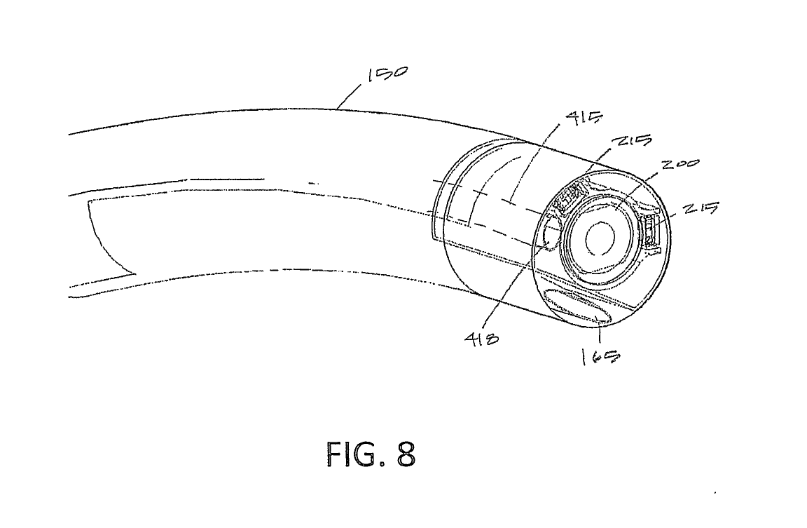

FIGS. 7A-7B illustrate another feature of the invention which relates to a pressure sensing subsystem that communicates with fluid management system 125 for maintaining a set pressure in a body cavity, such as a uterine cavity in a hysteroscopic procedure. Referring to FIG. 7A, the proximal hub portion 144 of the single-use endoscope component 110 is shown in an enlarged view. It can be seen that the proximal face 390 of the hub 144 includes projecting elements 400 and 405 that are adapted to connect with, or interface with, cooperating elements in handpiece 106. The upper projecting element 400 comprises a male-type electrical connector which couples all the electrical leads from the image sensor 200 and LEDs 215 to the handpiece processors and power source. The lower projecting element 405 is a pressure transducer and comprises a substantially rigid rectangular frame structure 406 around an interior chamber 408 wherein the inferior surface of the element 405 comprises an elastomeric transducer membrane 410. In other words, the elastomeric transducer membrane 410 is fitted over the interior chamber 408 and can flex as pressure changes in the chamber. The interior chamber 408 communicates with a open passageway 415 that extends through the endoscope shaft 150 to an open termination 418 in the distal portion of the shaft 150 (see FIG. 8). Thus, the transducer membrane 410 is in direct communication through passageway 415 with a working space 172 (FIG. 1) during a procedure as the distal end of the endoscope shaft 150 is immersed in a distention fluid.

FIG. 7B illustrates the single-use endoscope component 110 of FIG. 7A after being coupled to handpiece 106 which is shown partly in phantom view. The handpiece 106 includes a rigid member 420 that is adapted to interface with transducer membrane 410 of the hub 144. More in particular, the rigid member 420 carries first and second pressure sensors 425A and 425B that can comprise cylindrical wall elements and with an open interior chamber in each that is interior of an sensor membranes. The pressure sensors thus interface with the transducer membrane 410 of the single-use endoscope component 110. It can be understood how the transducer 415 then can transmit pressure signals to the pressure sensors. The two sensors are coupled to independent electronic sensor components in the handpiece 106 to provide for redundancy. The sensor signals then can be wirelessly transmitted from the handpiece 106 to the base station 120 and controller of the fluid management system 125 to control fluid inflows and outflows to, turn, control and maintain a set pressure.

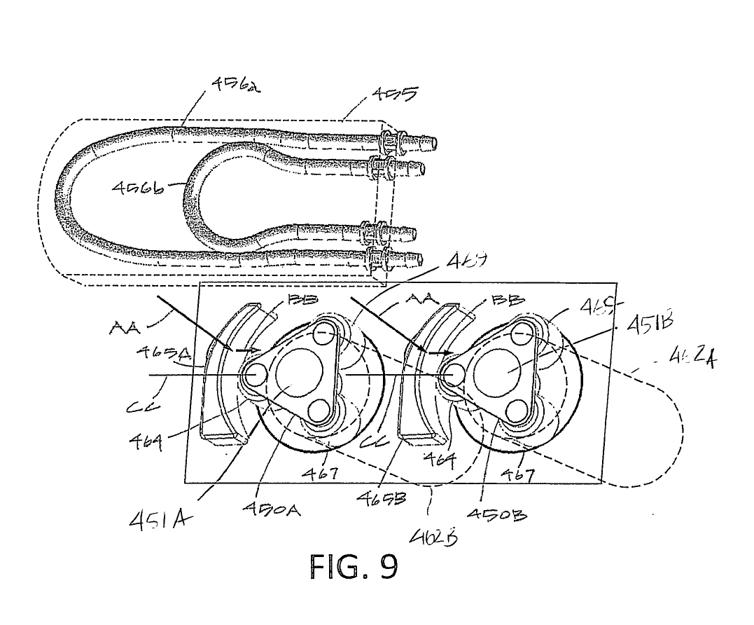

FIG. 9 shows another aspect of the invention relating to the fluid management system 125. In one variation, the base station 120 (FIG. 1), or an independent console, carries a pump head assembly including first and second peristaltic or roller pumps 450A and 450B to provide fluid inflows and outflows, respectively. Each roller pump typically comprises a rotor 451A and 451B, respectively, carrying a plurality of individual rollers, typically three rollers 464, 467, and 469 on each rotor, disposed to be rotationally advanced past a pair of stationary backing surfaces or “eyebrows” 465A and 465B to complete the pump head assembly.

A tubing cassette 455 (shown in phantom in FIG. 9) carries an inflow tubing loop 456a and an outflow tubing loop 456b adapted for coupling with the first roller pump 450A and second roller pump 450B, respectively. Typically, the tubing cassette 455 is first pushed over the pump head assembly (in direction of arrow AA) so that tubing loops 456A and 456B are inserted over the roller pumps 450A and 450B, respectively. Thereafter, the tubing cassette 455 is pushed in the direction of arrow BB to a “locked” position on the pump head assembly where the tubing loops 456A and 456B are compressed between the eyebrows 465A and 465B and the rollers 464, 467, and 469 of the roller pumps 450A and 450B. Thus, rotation of the rotors 451A and 451B will advance or pump fluids through the tubing loops 456a and 456b, respectively.

In one variation, the motors 462A and 462B (shown in phantom) for the pumps 450A and 450B comprise encoding-type motors (motors having integrated encoders) that are adapted to continuously send signals to a controller relating to the rotational position of the motor shaft. While motors having integrated encoders are disclosed, the present invention could use other encoders, such as optical external encoders, configured to track the rotational position of a motor shaft or of the pump rotors 451A and 451B themselves. Thus, the controller processor always knows the rotational position of the motor shaft and the rollers 464, 467, and 469 on each pump (since the motor shaft is directly connected to the pump rotor 451A and 451B which rotationally advances the individual rollers).

In another variation, the controller is adapted to rotate the rotors 451A and 451B on the roller pumps 450A and 450B to a preselected “stop” position at a pre-selected time(s). As can be seen in FIG. 9, the roller assemblies of each pump 450A and 450B are positioned such that a single roller 464 from each pump is aligned with the midpoint or centerline CC of the eyebrows 465A and 465B. It has been found that aligning a single roller 464 in each pump in the centerline position of FIG. 9 allows for a greatly reduced force to move the cassette from the “insertion” position to the “locked” position (see arrow BB) so that the tubing loops 456a, 456b engage the roller pumps. It can be understood that if the rollers 464a and 467a were rotated away from the position shown in FIG. 9, then the insertion force in the direction of arrow BB would significant increase since the tubing loops 456a, 465b would need to be compressed against two rollers 464 and 467 instead of a single roller 464.

The locking mechanism for maintaining cassette 455 in the “locked” position can be any one of a variety of tab-type features in the surface of the base station that engage the cassette 455. In one variation, the controller rotates the roller pumps to the position shown in FIG. 9 each time the cassette 455 is removed from the base station 120. Thus, the rollers 464, 467, and 469 would be in the desired preselected position as shown in FIG. 9 when a cassette 455 was subsequently loaded onto the roller pumps 450A and 450B. In another variation, an actuator button on the base station 120 could activate the motors to move the roller pumps to the preselected position prior to assembling the cassette 455 with the fluid management system. In another variation, each time the base station is set up for use, e.g. each time the pump head is powered, the controller could move the roller pumps to the preselected position.

In another aspect of the invention, during use of the system with the cassette in place, the controller can stop rotation of either or both pump shafts in the positions shown in FIG. 9 to pinch the tubing closed as a valve which may be useful during a procedure, for example to maintain fluid pressure in a working space.

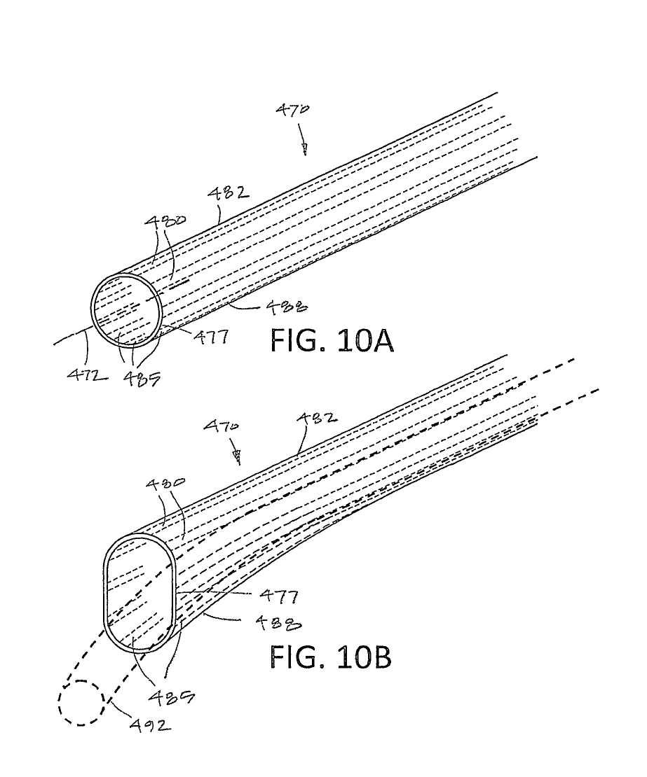

FIG. 10 illustrates another component of the invention which comprises an elastomeric working channel 470 that is configured for deformation or stretching in one radial direction away from axis 472 as opposed to stretching equally in 360°. In one variation, the walls of the working channel 470 comprise an elastomeric material 477 and relatively stiff ribs 480 on the superior surface 482 of the working channel and relatively flexible ribs 485 on the inferior surface 488 of the working channel. Thus, if an articulating shaft instrument 492 is inserted through the working channel 470, and the shaft is then articulated, the working channel 470 of FIG. 10 will stretch to accommodate the articulated shaft. In general, the invention comprises a deformable working channel 470 for use in an endoscope or introducer that has a skeleton of ribs that have differing flex properties embedded in an elastomeric surface to thus allow preferential radial stretching of the working channel in a selected direction rather than stretching similarly in 360°.

In another aspect of the invention, referring to FIGS. 7A-7B, the fluid outflow channel 494 through the endoscope shaft 150 terminates in a fitting 490 in the hub 144. The outflow tubing 495 which extends to a collection reservoir has a connector 496 that is adapted for detachable coupling to the fitting 490 in the hub 144. The fitting 490 comprises a closing valve 498 that closes when the outflow tubing connector 496 is detached from the fitting 490. The outflow tubing connector 496 opens the valve 498 when the connector 496 is connected to the fitting 490.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A fluid management system for use with a fluid source, a fluid receptacle, and a surgical tool having an inlet port for receiving fluid from the fluid source and an outlet port for discharging fluid to the fluid receptacle, said fluid management system comprising:
(a) a pump head assembly comprising:
an inlet pump having a rotor which carries a plurality of rollers, an arcuate roller backing adjacent the rotor to define a tube-receiving channel therebetween, and a rotor position sensor;
an outlet pump having a rotor which carries a plurality of rollers, an arcuate roller backing adjacent the rotor to define a tube-receiving channel therebetween, and a rotor position sensor;
an inlet motor configured to rotate the inlet pump rotor; and
an outlet motor configured to rotate the outlet pump rotor;
(b) a tubing cassette comprising:
an inlet tubing loop configured to be received about the rollers in the tube receiving channel of the inlet pump when the cassette is placed over the pump head assembly;
an outlet tubing loop configured to be received about the rollers in the tube receiving channel of the outlet pump when the cassette is placed over the pump head assembly;
(c) a controller configured to receive rotational position information from the rotor position sensor of each pump and to instruct the motor of each pump to drive said rotor and selectively stop each rotor at a preselected rotational position.

2. The fluid management system of claim 1, wherein said sensor comprises an encoder.

3. The fluid management system of claim 2, wherein said encoder is formed integrally in the motor.

4. The fluid management system of claim 2, wherein said encoder is externally mounted to the motor or rotor.

5. The fluid management system of claim 1, wherein the controller is configured to selectively stop at least one rotor so that one roller is aligned with a centerline of the arcuate roller backing.

6. The fluid management system of claim 1, wherein the controller is configured to selectively stop both rotors so that each rotor has a roller aligned with a centerline of the adjacent arcuate roller backing.

7. The fluid management system of claim 1, wherein the pumps and the controller are housed together in a single enclosure.

8. A method for loading a tubing cassette on a pump head assembly, said method comprising:
(a) providing a pump head assembly comprising:
an inlet pump having a rotor, a plurality of rollers, and an arcuate roller backing adjacent the rotor to define a tube receiving channel therebetween;
an outlet pump having a rotor, a plurality of rollers, and an arcuate roller backing adjacent the rotor to define a tube receiving channel therebetween;
an inlet motor configured to rotate the inlet pump rotor; and
an outlet motor configured to rotate the outlet pump rotor;
(b) providing a tubing cassette comprising:
an inlet tubing loop configured to be received about the rollers in the tube receiving channel of the inlet pump when the cassette is placed over the pump head assembly;
an outlet tubing loop configured to be received about the rollers in the tube receiving channel of the outlet pump when the cassette is placed over the pump head assembly;
(c) causing the rotors on the pump head to each align one roller with a centerline of the adjacent arcuate roller backing, wherein no other rollers are in the tube-receiving channel; and
(d) placing the cassette over the pump head assembly so that both the inlet tubing loop and the outlet tubing loop are received in the tube-receiving channels of the inlet and outlet pumps, respectively.

9. The method of claim 8, wherein causing the rotors on the pump head to each align one roller with a centerline of the adjacent arcuate roller comprises sensing a position of the rotors and automatically aligning the rotors.

10. The method of claim 9, wherein automatically aligning the rotors comprises aligning the rotors each time the cassette is removed from the pump head.

11. The method of claim 10, wherein aligning the rotors each time the cassette is removed from the pump head is performed by a controller.

12. The method of claim 9, wherein automatically aligning the rotors comprises manually instructing a controller to automatically align the rotors.

13. The method of claim 9, wherein automatically aligning the rotors comprises a controller automatically aligning the rotors each time the pump head is powered.

14. The method of claim 9, wherein sensing the position of the rotors comprises reading an encoder coupled to the motor or the rotor.
